(19) United States
(12) Patent Application Publication
(10) Pub. No.: US 2002/0104856 A1
(43) Pub. Date: Aug. 8, 2002

(54) Oral medicine dispenser

(76) Inventors: Malcolm David Clark, Barnet (GB); Bruce Cameron Croft, Cranbrook (GB); George Calvin Davies, Chomberg (CA)

Correspondence Address: GALGANO & BURKE, 300 Rabro Drive, Suite 135, Hauppauge, NY, 11788, US

(21) Appl. No.: 10/026,852

(22) Filed: Dec. 21, 2001

Related U.S. Application Data
(63) Continuation-in-part of application No. PCT/GB00/00821, filed on Mar. 8, 2000.

(30) Foreign Application Priority Data
Jun. 23, 1999 (GB) 9914621.9

(51) Int. Cl. A61M 5/178; A61J 7/00; B65D 35/00; B65D 37/00; B05B 7/00; B05B 11/06

(52) U.S. Cl. 222/633; 222/630; 222/92; 604/37; 604/77

(57) ABSTRACT

The present invention provides an oral medicine dispenser for the administration of a single pre-measured dose, which dispenser comprises a body having a collapsible reservoir portion and an elongate dispensing tube portion extending from the reservoir portion, wherein the reservoir portion holds, in use, a single dose of a fluid oral medicament and the dispensing tube portion has an end cap, whereby on opening of the end cap the collapsible reservoir portion may be manually squeezed to collapse the reservoir portion sufficiently to dispense substantially all of, or at least a major part of the medicament dose through the dispensing tube portion.

### FIELD OF THE INVENTION

The present invention relates to an oral medicine dispenser that is adapted for administration of a single pre-measured dose.

### BACKGROUND TO THE INVENTION

Administration of liquid oral medicaments to small children, the elderly and infirm can be a difficult task to execute correctly using the conventional measuring spoon and supply of medicament in a tamper-proof or tamper-evident bottle. Not only is there the difficulty of accurately filling the spoon to the required dose but more problematically, there is the substantial risk that the patient may not properly consume the dose held in the head of the spoon. Particularly with children, spillages are frequent.

Further problems with the use of a bottle of medicament and dosing spoon are the need for inclusion of preservatives in the medicament which in turn leads to risk of allergenic sensitisation against the preservatives, especially in young children and babies.

Among the many further limitations of the conventional bottle and spoon are the difficulties of making the container effectively tamper evident, keeping costs to a minimum and avoiding build-up of residues around the neck of the bottle. Many liquid oral medicaments are in syrup form and have a tendency to congeal around the bottle neck.

Prior art proposals for alternatives to the conventional bottle and spoon have included disposable single pre-measured dose containers. One example of such a container is illustrated in European Patent Application EP 0 778 018 A1 and comprises a spoon-shaped peel-off container having a hollow thermo-formed body holding the single dose of medicament and which is sealed in by a peel-off film or foil. The body has a short flat extension for use as a handle.

A similar device for administration of a single pre-measured dose is illustrated in UK Patent Application GB 2 306 322 A and comprises a spatula-like element having a rupturable medicament container fastened to the base of the device. The container is ruptured upon withdrawing the device between the teeth or lips of the patient to disgorge the contents into the mouth of the patient.

Whereas these prior art devices partially address the problems associated with the conventional bottle and spoon they do not resolve such problems as the risk of spillage of the medicament and not giving full control over administration to the holder of the device, amongst others.

It is the general objective of the present invention to provide an improved oral medicine dispenser for administration of a single pre-measured dose and which mitigates against or overcomes some or all of the aforementioned and other problems of the prior art.

### SUMMARY OF THE INVENTION

According to the present invention, there is provided an oral medicine dispenser containing and for administering a single pre-measured dose, which dispenser comprises a body having a collapsible reservoir portion and an elongate dispensing tube portion extending from the reservoir portion, wherein the reservoir portion holds a single dose of a fluid oral medicament and the dispensing tube portion has an end cap, whereby on opening of the end cap the collapsible reservoir portion may be manually squeezed to collapse the reservoir portion sufficiently to dispense substantially all of, or at least a major part of, the medicament dose through the dispensing tube portion wherein the device is shaped to resemble a spoon, at least in profile.

The end cap of the dispensing tube portion is suitably a frangible or severable end part of the dispensing tube portion.

The dispenser is preferably sufficiently transparent to enable viewing of its contents although it may be coloured—for example colour coded to reflect aspects of its medicament contents. It is preferably of substantially unitary construction and is suitably formed of a plastics material by a form, fill and seal technique in which a plastics material is formed to substantially the required shape and with an opening to introduce the fluid oral medicament, the opening then being sealed.

It will be noted that the device is shaped to resemble a spoon, at least in profile. That part of the dispenser which resembles the head of a spoon is the reservoir portion and that part of the dispenser which resembles the handle of the spoon is the dispensing tube portion. In practical terms, however, that part which resembles the handle of a spoon does not have the function of a handle. The primary mode of use of the dispenser involves handling it by the reservoir portion for the dispensing of the medicament. The purpose of forming the dispenser to resemble a spoon is not that it may perform the function of a spoon but rather that the profile shape is surprisingly convenient for the purpose of squeeze-dispensing the medicament. Indeed, the reservoir portion provides for a more secure handle than that of a spoon. Furthermore, the similarity of appearance to the shape of a spoon is comfortingly reassuring to the patient.

The reservoir portion of the dispenser is preferably generally hemispherical or at least having a substantially flat wall on one face and an opposing substantially domed wall. This flattened bulb shape ties in well with the spoon-shaped profile and also facilitates manufacture, reducing spillage during filling of the dispenser in manufacture while enabling the reservoir to be relatively easily squeezed to collapse and dispense the medicament.

The internal wall(s) of the reservoir portion is/are suitably rounded to mitigate against the risk of entrapment of any medicament.

The dispenser of the present invention is economical to manufacture and simple, fun and straightforward to use with minimal spillage and minimal residue and is especially suitable for administration of syrupy medicaments to infants, the elderly and the infirm.

In a further improvement to the dispenser, the dispenser suitably has at least a further reservoir portion linked to said reservoir portion by a tube portion that is preferably the dispensing tube portion. This enables the user to readily select between different dose options. The end cap may comprise the further reservoir portion.

In one aspect of the present invention there is provided an oral medicine dispenser containing and for administering a single pre-measured dose, which dispenser comprises a body having a collapsible reservoir portion and an elongate dispensing tube portion extending from the reservoir portion, wherein the reservoir portion holds a single dose of a fluid oral medicament and the dispensing tube portion has an end cap, whereby on opening of the end cap the collapsible reservoir portion may be manually squeezed to collapse the reservoir portion sufficiently to dispense substantially all of, or at least a major part of, the medicament dose through the dispensing tube portion ,the end cap comprising a further reservoir portion of a different capacity to said reservoir portion whereby the user may select the dose to be dispensed.

### BRIEF DESCRIPTION OF THE DRAWINGS

Preferred embodiments of the present invention will now be more particularly described, by way of example, with reference to the accompanying drawings, wherein:

FIGS. 1 and 2 are, respectively, a side elevation view and a top plan view of the first preferred embodiment of dispenser;

FIG. 3 is a longitudinal sectional view of the first preferred embodiment; and

FIGS. 4 and 5 are a side elevation view of a second embodiment and side perspective view of a third embodiment, respectively.

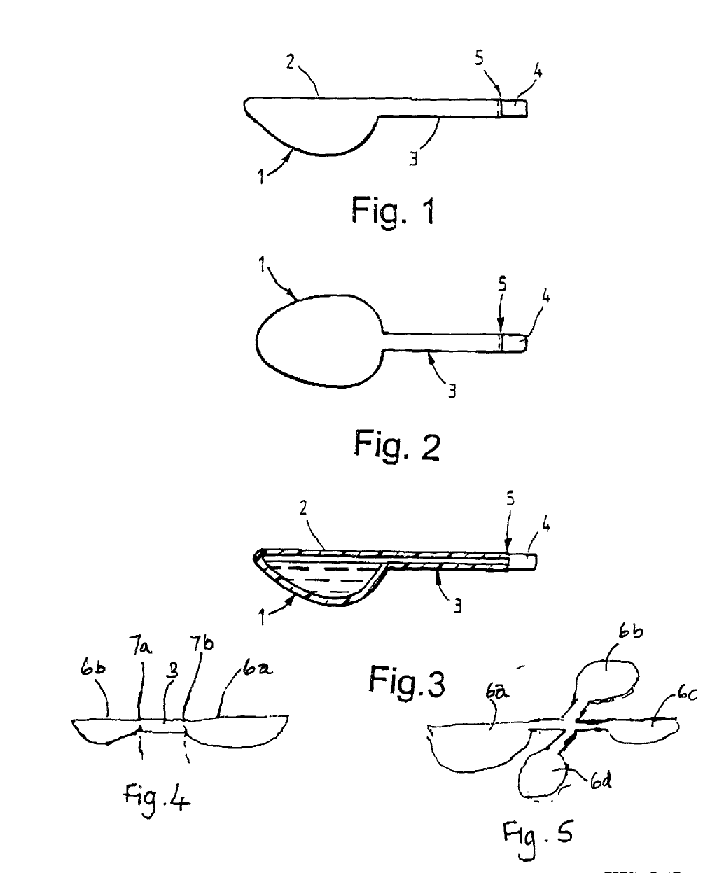

### DESCRIPTION OF THE PREFERRED EMBODIMENT

As will be seen from the accompanying figures, the preferred embodiment has the general shape of a spoon when viewed in profile and, for that matter, having the outline of a spoon when viewed in plan. However, as may be clearly seen from the sectional view, that portion of the dispenser which resembles the head of the spoon is a reservoir bulb 1 having a flat upper surface 2. That portion of the dispenser which resembles the handle of a spoon is a dispensing tube 3.

The preferred embodiment of dispenser illustrated is formed by a blow-fill and seal technique in which low density polyethylene, or similar, polymer beads are melted and extruded and during the extrusion blown into the required form with nitrogen gas or other, preferably inert, gaseous medium. An opening is left in the moulding to allow the oral medicament to then be introduced into the hollow interior of the dispenser. For obvious reasons, this opening is suitably formed in the upper surface of the dispenser moulding, containing the liquid oral medicament therewithin until the opening is sealed.

The entirety of the dispenser is formed in this single moulding process. Accordingly, the reservoir bulb portion 1 and dispensing tube portion 3 have a unitary construction.

Provision is made for straightforward opening of the tip 4 of the dispensing tube portion 3 by snapping/tearing, cutting or otherwise detaching or releasing the end of the tubular portion 3. In its simplest form, this may be achieved by a thinning of the moulding at a suitable pre-breakage line 5 extending around the “circumference” of the tubular portion 3. This pre-breakage line may be introduced as a relatively thinner section of the tubular portion during the moulding process or may be subsequently scored onto the moulding.

The thus-formed dispenser suitably has a capacity equivalent to that of any conventional dose measuring spoon and which may deliver of the order of 2.5-10 ml of the medicament and preferably of the order of 5 ml.

The construction of the reservoir bulb portion 1 is such as to enable it to relatively straightforwardly be squeeze compressed between thumb and forefinger to reliably expel substantially the whole contents of the reservoir bulb portion 1 and tubular portion 3 through the opened end of the tubular portion 3. Little medicament residue need be left behind and the dispenser may be disposed of following use.

The action of removing the end portion/cap or tip 5 of the dispenser is perhaps most conveniently carried out by holding the tubular portion 3 close to the score-line 4 and with the reservoir bulb portion 1 lowermost and then simply snapping or cutting off the end portion of the dispensing tube portion 3 of the dispenser.

During construction of the dispenser it may be desirable to reinforce the tubing portion 3 by, for example, configuring the moulding so that the tubular dispensing portion 3 has a somewhat thicker cross-section than the reservoir bulb portion 1 to give it greater rigidity than the reservoir bulb portion 1. The reservoir portion 1 must inherently have an adequate flexibility to optimise the dispensing action but this is less important for the dispensing tube portion 3 and it is useful to be able to grip the tube portion 3 firmly without squeezing it when severing the tip 4.

The volume of medicament enclosed within the dispenser may suitably be slightly greater than the required dose to allow for any part of the dispensing tube portion 3 that may not be fully collapsible.

In an improvement to the design of the dispenser as illustrated in FIGS. 4 and 5, to enable a user to simply and effectively titrate their own appropriate dose, the collapsible reservoir 1 of the dispenser may be linked via the tube portion 3 to one or more other reservoirs 6.

In FIG. 4 the dispenser has a large reservoir portion 6a of 5 ml capacity and which is linked to a smaller reservoir portion 6b of 2.5 ml capacity. The dispenser may thus be pre-filled to contain 5 ml, giving the user the choice of consuming the full 5 ml by ensuring that the content is fully transferred into the larger reservoir portion 6a before breaking the tube portion 3 at a breakage line 7a adjacent the smaller reservoir portion 6b; or selecting and consuming only 2.5 ml, simply by squeezing or tilting the dispenser so that the smaller reservoir portion 6b is full prior to breaking the tube portion 3 at a breakage line 7 adjacent the larger reservoir portion 6a. This arrangement gives the user choice and extends the number of intermediate doses available, eg 2.5 ml, 5 ml, 7.5 ml etc.

Referring to FIG. 5, the design may be developed to give multiple reservoirs 6a-d each of a different capacity to give the desired greater choice in dose selection. The largest reservoir portion 6a is filled to capacity and the user selects his dose by orientating the dispenser appropriately and squeezing to fill the desired reservoir. The desired reservoir is bent downwardly to minimise spillage and may be twisted to shear and separate giving the required dose. This configuration of dispenser may be spoon-shaped or have reservoir portions of different shapes, diameters/volumes

1. An oral medicine dispenser containing and for administering a single pre-measured dose, which dispenser comprises a body having a collapsible reservoir portion and an elongate dispensing tube portion extending from the reservoir portion, wherein the reservoir portion holds a single dose of a fluid oral medicament and the dispensing tube portion has an end cap, whereby on opening of the end cap the collapsible reservoir portion may be manually squeezed to collapse the reservoir portion sufficiently to dispense substantially all of, or at least a major part of, the medicament dose through the dispensing tube portion wherein the device is shaped to resemble a spoon, at least in profile.

2. An oral medicine dispenser as claimed in claim 1 wherein at least the reservoir portion is at least sufficiently transparent to view the contents thereof.

3. An oral medicine dispenser as claimed in claim 1, wherein the end cap of the dispensing tube portion is a frangible or severable end part of the dispensing tube portion.

4. An oral medicine dispenser as claimed in claim 1, wherein the dispenser is of substantially unitary construction.

5. An oral medicine dispenser as claimed in claim 4, wherein the dispenser is formed of a plastics material by a form, fill and seal technique.

6. A dispenser as claimed in claim 1, wherein the reservoir portion of the dispenser is generally hemispherical or at least having a substantially flat wall on one face and an opposing substantially domed wall.

7. A dispenser as claimed in claim 1, having at least a further reservoir portion linked to said reservoir portion by a tube portion

8. A dispenser as claimed in claim 7, wherein the tube portion is the dispensing tube portion.

9. A dispenser as claimed in claim 8,wherein the end cap omprises the further reservoir portion.

10. An oral medicine dispenser containing and for administering a single pre-measured dose, which dispenser comprises a body having a collapsible reservoir portion and an elongate dispensing tube portion extending from the reservoir portion, wherein the reservoir portion holds a single dose of a fluid oral medicament and the dispensing tube portion has an end cap, whereby on opening of the end cap the collapsible reservoir portion may be manually squeezed to collapse the reservoir portion sufficiently to dispense substantially all of, or at least a major part of, the medicament dose through the dispensing tube portion, the end cap comprising a further reservoir portion of a different capacity to said reservoir portion whereby the user may select the dose to be dispensed.
